(19) United States
(12) Patent Application Publication
(10) Pub. No.: US 2006/0293591 A1
(43) Pub. Date: Dec. 28, 2006

(54) Implantable medical device with MRI and gradient field induced capture detection methods

(76) Inventors: John D. Wahlstrand, Shoreview, MN (US); Greg A. Younker, White Bear Township, MN (US); Lawrence C. McClure, Forest Lake, MN (US); Kent Samuelson, Parker, CO (US); Robert T. Sawchuk, Vadnais Heights, MN (US)

Correspondence Address: MEDTRONIC, INC., 710 MEDTRONIC PARK, MINNEAPOLIS, MN, 55432-9924, US

(21) Appl. No.: 11/127,352

(22) Filed: May 12, 2005

(51) Int. Cl. A61B 5/05

(52) U.S. Cl. 600/423

(57) ABSTRACT

An implantable medical device is provided having a telemetry circuit antenna; a lead having an elongated body for carrying a conductor extending from a proximal connector to a distal electrode; a circuit for measuring voltage induced on the telemetry circuit antenna and generating an antenna voltage signal corresponding to the measured voltage on the antenna; a circuit for measuring voltage induced on the lead conductor and generating a lead voltage signal corresponding to the measured voltage on the lead; and processing circuitry for receiving the antenna voltage signal and the lead voltage signal and for generating an MRI detection signal if the antenna voltage signal and the lead voltage signal meet an MRI detection requirement. The device further includes control circuitry for providing a safeguard response to the MRI detection signal.

### FIELD OF THE INVENTION

The present invention relates generally to implantable medical devices and in particular to a method and apparatus for detecting electromagnetic interference (EMI) due to magnetic resonance imaging (MRI) equipment and for providing a safeguard response to the detection.

### BACKGROUND OF THE INVENTION

Patients having an implantable medical device (IMD) may be submitted to MRI examinations for a variety of reasons. Exposure to the strong magnetic field can cause electromagnetic interference (EMI) that can cause improper device function. EMI caused by exposure to an MRI magnetic field can arise on the telemetry antenna included in programmable IMDs and on the leads carrying sensing and stimulation electrodes extending from the IMD. IMDs which provide electrical stimulation therapies, such as neurostimuators, cardiac pacemakers and implantable cardioverter defibrillators (ICDs) may inappropriately detect magnetic field induced signals as physiological signals. Furthermore, magnetic field induced current on lead conductors may result in inappropriate stimulation or heating.

In past practice, an IMD may be programmed prior to an MRI examination to prevent undesired results of EMI. For example, an ICD may be programmed to disable arrhythmia detection prior to an MRI examination. However, this does not prevent inadvertent tissue stimulation due to induced current on stimulation leads. Furthermore, an IMD programmer and personnel skilled in programming an IMD may not be readily available at the MRI facility. A pacemaker or ICD patient may need to visit a cardiology clinic prior to his/her MRI examination to have the IMD programmed and then return to the cardiology clinic after the MRI examination to have the IMD re-programmed. Scheduling conflicts could result in delays between the programming sessions and the MRI examination which could leave the IMD functioning in a less than optimal operating mode, potentially for several days. The patient may be left vulnerable to clinical events or conditions normally controlled or treated by the IMD. For these reasons, it is important to safeguard against the effects of the strong magnetic field on an IMD and associated leads during an MRI examination.

### BRIEF SUMMARY OF THE INVENTION

The present invention provides an IMD having MRI field detection circuitry and an associated method for detecting the presence of an MRI field and providing a safeguard response. The IMD includes: telemetry circuitry with a telemetry antenna used for wireless communication with an external programmer or monitoring device; one or more associated leads for deploying electrodes at a tissue stimulation or sensing site; sensing circuitry for measuring voltage signals on the telemetry antenna and one or more lead conductors; processing circuitry for comparing measured voltage signals to predetermined threshold levels, and for generating an MRI detection signal when the measured telemetry antenna and lead voltage signals both meet MRI detection requirements; and control circuitry for responding to the MRI detection signal.

The associated method includes sampling the telemetry antenna voltage and the lead voltage at desired sampling rates and providing signals corresponding to the respective telemetry antenna and lead voltages. The telemetry antenna voltage and the lead voltage can be measured by sampling onto capacitors and converting the resulting capacitor voltages to digital values. The digital voltage values are compared to MRI detection thresholds defined for the antenna voltage and the lead voltage signals. An MRI detection requirement is predefined based on the frequency and number of MRI detection threshold crossings. If the MRI detection requirement is satisfied, the IMD control circuitry provides a response. Responses may include generating an alarm and/or implementing a set of temporary operating parameters. In one embodiment, an MRI detection response includes a capture test for determining if the energy associated with the induced lead voltage is high enough to capture excitable tissue in contact with an electrode carried by the lead.

### BRIEF DESCRIPTION OF THE DRAWINGS

FIG. 1 is an illustration of an IMD implanted in a patient's body.

FIG. 2 is a block diagram of typical functional components of an IMD, such as IMD 10 shown in FIG. 1.

FIG. 3 is a functional block diagram of an MRI field detector according to one embodiment of the present invention.

FIG. 4 is a flow chart summarizing steps included in a method for detecting an MRI field and providing a safeguard response.

FIG. 5 is a flow chart summarizing steps included in one method for implementing an MRI safe operating mode.

FIG. 6 is a flow chart summarizing steps included in an alternative method for implementing MRI-safe operating parameters in response to an MRI detection.

### DETAILED DESCRIPTION

The invention is directed toward providing an implantable medical device with the capability of detecting the presence of a strong magnetic field associated with an MRI environment. The invention is further directed toward providing an automatic safeguard response to the detected presence of a magnetic field. Automatic implementation of temporary, “MRI-safe” parameter settings by the IMD in the presence of the MRI environment and the reversal to permanently programmed values outside of the MRI environment allows the “MRI-safe” operation of the IMD to be limited to the time of the MRI examination, while the patient is under medical supervision.

Aspects of the present invention can improve the safety and performance of any IMD that includes electrical stimulation or sensing of electrical body signals. Such devices include drug pumps, cardiac stimulation devices such as pacemakers and implantable cardioverter defibrillators, and other neuromuscular stimulators, such as deep brain stimulators, spinal cord stimulators, stimulators used for treatment of sleep apnea, fecal or urinary incontinence, smooth muscle stimulators used for treating digestive tract disorders, function electrical stimulation devices, vagal nerve stimulators, and diaphragm stimulators. By providing a safeguard response to a detected magnetic field, inadvertent tissue stimulation due to induced current on stimulation leads can be prevented. The likelihood of inappropriate detection of EMI as physiological signals, which may cause an inappropriate therapy response, can be avoided. A patient having an IMD for treating one medical condition may undergo MRI for another, unrelated medical condition. The clinician prescribing the MRI may therefore not be fully aware of the clinical implications of exposing the IMD to an MRI field. Incorporation of the automatic detection of and safeguard response to a magnetic field within the IMD assists clinicians in ensuring the safety of the IMD patient, regardless of differences in medical specialties between the IMD-prescribing clinician and the MRI-prescribing clinician.

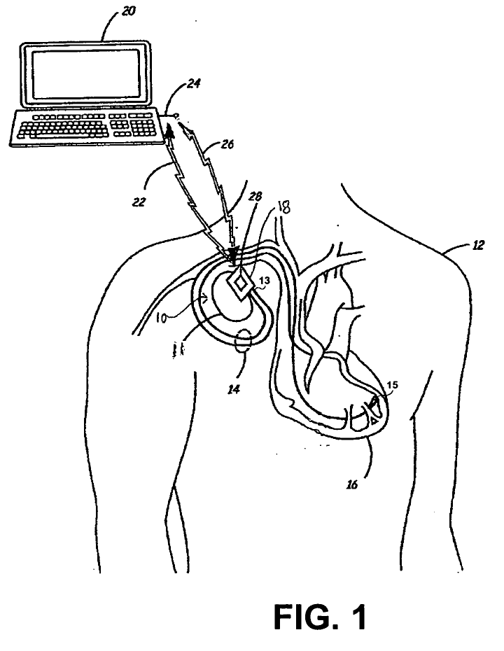

FIG. 1 is an illustration of an IMD implanted in a patient's body. IMD 10 is depicted as a cardiac stimulation device for the sake of illustrating one type of electrical stimulation and sensing IMD in which the aspects of the invention may be implemented. FIG. 1 is provided to illustrate one type of IMD in which the invention can be incorporated and is not intended to limit the scope of the invention to cardiac stimulation devices or a particular type of cardiac stimulation device.

IMD 10 is implanted in a patient 12 beneath the patient's skin or muscle and, in this example, is electrically coupled to the heart 16 of the patient 12 through pace/sense electrodes 15 and lead conductor(s) of one or more associated cardiac pacing leads 14 in a manner known in the art. Leads 14 include a conductor extending from a proximal connector 13 adapted for connection to IMD 10 to the distal electrodes 15. Alternatively, subcutaneous electrodes may be employed, thereby eliminating the leads from the device to the heart. IMD 10 is capable of telemetric communication with an external medical device 20, typically embodied as a programmer or monitor in a manner known in the art.

Programming commands or data can be transmitted between an IMD telemetry antenna 28 and an external telemetry antenna 24 associated with the external programmer 20 using, for example, RF transmission or other wireless communication modalities. In an uplink telemetry transmission 22, the external telemetry antenna 24 operates as a telemetry receiver antenna, and the IMD telemetry antenna 28 operates as a telemetry transmitter antenna. Conversely, in a downlink telemetry transmission 26, the external telemetry antenna 24 operates as a telemetry transmitter antenna, and the IMD telemetry antenna 28 operates as a telemetry receiver antenna. Both telemetry antennas are coupled to transceiver circuitry including a transmitter and a receiver.

IMD telemetry antenna 28 is generally designed for efficient, reliable telemetry transmission in the implanted environment. IMD telemetry antenna 28 may be located within the hermetic IMD housing 11 containing the device circuitry, in or on a plastic header or connector block 18 used to interconnect the IMD 10 to electrical leads 14, mounted to the IMD housing 11, or incorporated as a portion of one of the electrical leads 14. When located outside the IMD housing 11, IMD telemetry antenna 28 is coupled to transceiver circuitry within the housing 11 of IMD 10 via an insulated, conductive feed-through extending through the connector block 18. IMD telemetry antenna 28 is typically a monopole antenna having a length tuned to function optimally at the radio frequencies chosen for use in the telemetry system.

In the presence of a magnetic field associated with an MRI environment, IMD telemetry antenna 28 interacts with the MRI magnet to form an air-core transformer with the MRI magnet as the primary coil and the IMD telemetry antenna 28 as a multiple-winding secondary coil. An electrical lead 14 coupled to IMD 10 also interacts with the MRI magnet as a single-winding secondary coil. Voltage (V) can be induced on both the IMD telemetry antenna 28 and leads 14 in accordance with Faraday's Law:
V=n*(loop area)*dB/dt

wherein n is the number of turns of the IMD telemetry antenna 28 or the lead (n=1), the loop area is the area formed by IMD telemetry antenna 28 or the lead loop area, and dB/dt is the rate of change of the magnetic field strength.

In one example, the voltage induced on an IMD telemetry antenna having a loop area of approximately 2.77 cm² and 253 turns placed in a 3.0 Tesla magnet (dB/dt approximately 133 T/s in the body) could be greater than 9 Volts. Other EMI sources can induce similarly high voltages on the IMD telemetry antenna. Therefore, detection of a strong magnetic field associated with MRI based on monitoring IMD telemetry antenna voltages may not be reliable. As such, the invention provides MRI detection requirements relating to both IMD telemetry antenna voltage measurements and lead voltage measurements.

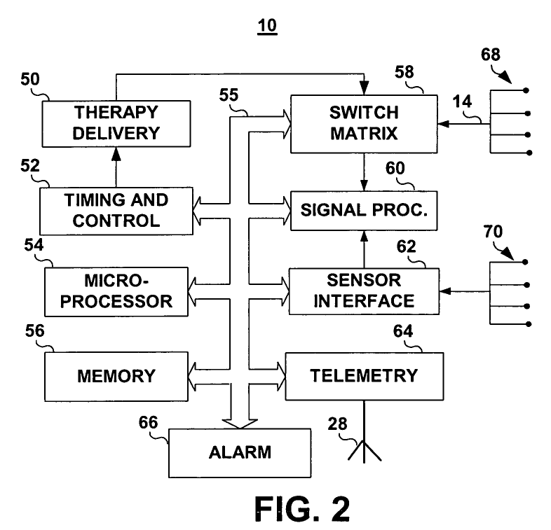

FIG. 2 is a block diagram of typical functional components of an IMD, such as IMD 10 shown in FIG. 1. IMD 10 generally includes timing and control circuitry 52 and an operating system that may employ microprocessor 54 or a digital state machine for timing sensing and therapy delivery functions in accordance with a programmed operating mode. Microprocessor 54 and associated memory 56 are coupled to the various components of IMD 10 via a data/address bus 55. IMD 10 may include therapy delivery unit 50 for delivering a therapy, such as an electrical stimulation or drug therapy, under the control of timing and control 52. In the case of electrical stimulation therapies, such as cardiac stimulation therapies, therapy delivery unit 50 is typically coupled to two or more electrodes 68 via a switch matrix 58. Switch matrix 58 is used for selecting which electrodes and corresponding polarities are used for delivering electrical stimulation pulses.

Electrodes 68 may also be used for sensing electrical signals within the body, such as cardiac signals or other electromyogram signals, or for measuring impedance. In the case of cardiac stimulation devices, cardiac electrical signals are sensed for determining when an electrical stimulation therapy is needed and in controlling the timing of stimulation pulses. Cardiac EGM sensing methods and arrhythmia detection and discrimination methods are known in the art.

Electrodes 68 are generally carried on one or more leads 14 coupled to IMD 10 as described in conjunction with FIG. 1. Electrodes used for sensing and electrodes used for stimulation may be selected via switch matrix 58. When used for sensing, electrodes 68 are coupled to signal processing circuitry 60 via switch matrix 58. Signal processor 60 includes sense amplifiers and may include other signal conditioning circuitry and an analog to digital converter. Electrical signals may then be used by microprocessor 54 for detecting physiological events, such as detecting and discriminating cardiac arrhythmias. Impedance signals can also be used for monitoring lead performance and detecting lead-related problems as is known in the art.

IMD 10 may additionally or alternatively be coupled to one or more physiological sensors 70. Such sensors may include pressure sensors, accelerometers, flow sensors, blood chemistry sensors, activity sensors or other physiological sensors known for use with IMDs. Sensors 70 are coupled to IMD 10 via a sensor interface 62 which provides sensor signals to signal processing circuitry 60. Sensor signals are used by microprocessor 54 for detecting physiological events or conditions. For example, IMD 10 may monitor heart wall motion, blood pressure, blood chemistry, respiration, or patient activity. Monitored signals may be used for sensing the need for delivering a therapy under control of the operating system.

The operating system includes associated memory 56 for storing a variety of programmed-in operating mode and parameter values that are used by microprocessor 54. The memory 56 may also be used for storing data compiled from sensed physiological signals and/or relating to device operating history for telemetry out on receipt of a retrieval or interrogation instruction. All of these functions and operations are known in the art, and many are generally employed to store operating commands and data for controlling device operation and for later retrieval to diagnose device function or patient condition.

IMD 10 further includes telemetry circuitry 64 and antenna 28. Programming commands or data are transmitted during uplink or downlink telemetry between IMD telemetry circuitry 64 and external telemetry circuitry included in a programmer or monitoring unit as described in conjunction with FIG. 1. Telemetry circuitry 64 and antenna 28 may correspond to telemetry systems known in the art.

For example, telemetry circuitry 64 may require the use of an external programming head containing an external antenna to be positioned over IMD 10 as generally disclosed in U.S. Pat. No. 5,354,319 issued to Wyborny et al. Long-range telemetry systems, which do not require the use of a programming head, are generally disclosed in U.S. Pat. No. 6,482,154 issued to Haubrich et al. Both patents are incorporated herein by reference in their entirety.

IMD 10 may optionally be equipped with patient alarm circuitry 66 for generating audible tones, a perceptible vibration, muscle stimulation or other sensory stimulation for notifying the patient that a patient alert condition has been detected by IMD 10. According to one embodiment of the present invention, an alarm signal is generated upon detection of an MRI field. Patient alarm circuitry 66 may be implemented according to alarm circuitry known in the art.

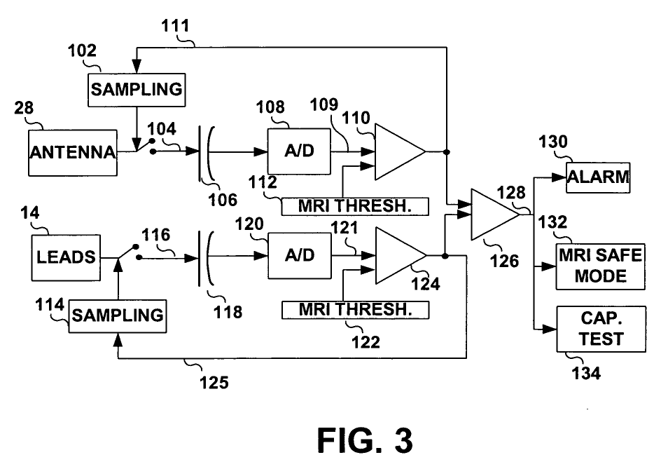

FIG. 3 is a functional block diagram of an MRI field detector according to the present invention. In order to detect an MRI field, induced voltages measured on both the IMD telemetry antenna 28 and on one or more conductors included in lead set 14 should exceed corresponding MRI detection threshold requirements. As such, the IMD telemetry antenna voltage is monitored on antenna voltage signal line 104, and one or more lead voltage signals are monitored on lead voltage signal line(s) 116. The antenna voltage is sampled at a selected sampling rate 102 onto a capacitor 106 and converted to a digital signal by A/D converter 108. The digitized antenna voltage signal 109 is provided to a comparator 110 which compares the antenna voltage signal 109 to an MRI detection threshold level 112.

The lead voltage signal 116 is sampled at a selected sampling rate 114 onto capacitor 118 and converted to a digital lead voltage signal 121 by A/D converter 120. The digitized lead voltage signal 121 is provided to a comparator 124 for comparison to an MRI detection threshold level 122. The sampling rate 102 for sampling the antenna voltage signal 14 and the sampling rate 114 for sampling the lead voltage signal 116 are controlled by microprocessor 54 (shown in FIG. 2). In one embodiment sampling rates 102 and 114 are increased in response to the detection of an MRI detection threshold crossing as indicated by feedback lines 111 and 125. One or both sampling rates 102 and 114 may be increased in response to a predetermined number of detected threshold crossings.

The outputs of comparators 124 and 110 are provided to an MRI detection module 126 which compares the frequency of MRI detection threshold crossings to an MRI detection requirement. When the MRI detection requirement is satisfied, an MRI detection signal 128 is generated. Microprocessor 54 will provide a response to an MRI detection signal 128 which may include any of generating an alarm signal 130, converting the IMD operating mode to a temporary MRI safe mode 132, or performing a capture test 134 for determining if the induced lead voltage signal 116 is likely to cause capture of excitable tissue at an electrode site. Comparisons performed by comparators 124 and 110 and by MRI detection module 126 may be implemented in firmware or software executed by microprocessor 54 (FIG. 2).

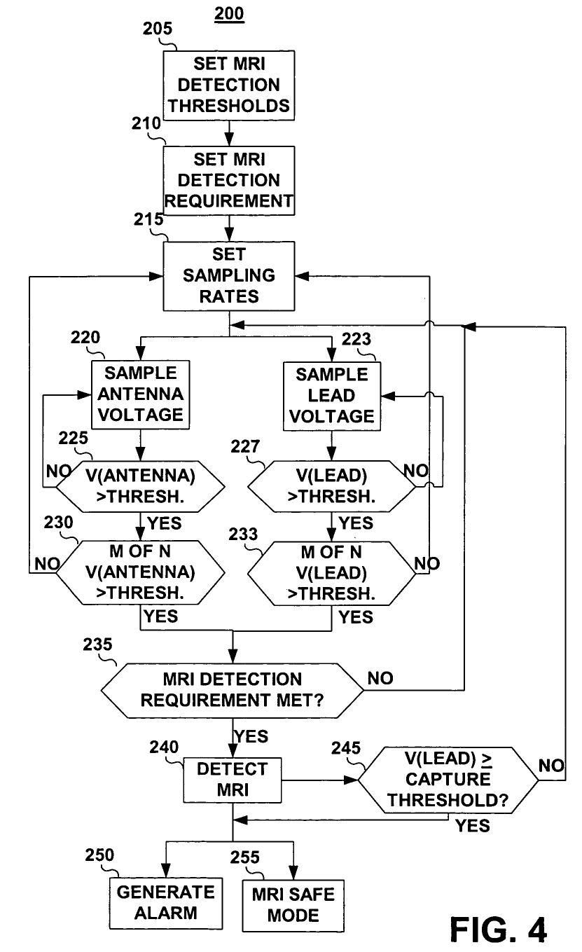

FIG. 4 is a flow chart summarizing steps included in a method for detecting EMI associated with an MRI environment and providing a safeguard response. At step 205, an MRI detection threshold is set for the telemetry antenna voltage signal, and an MRI detection threshold is set for the lead voltage signal. These thresholds are set uniquely from other EMI detection levels which may be included in EMI monitoring functions of IMD 10. For example, other sources of EMI may be related to electronic surveillance equipment or metal detectors, which may have separately defined detection thresholds.

An MRI detection requirement is set at step 210. In one embodiment, the detection requirement is based on detecting a required number of threshold crossings for both the antenna and lead voltage signals out of a predetermined number of sampled voltage measurements. In other embodiments, the MRI detection requirement may be based on a correlation between MRI detection threshold crossings occurring on the antenna voltage signal and on the lead voltage signal. Generally, the MRI detection requirement is selected so as to detect frequent high voltage signals on the telemetry antenna contemporaneous with frequent high voltage signals on one or more lead conductors. The voltage signal levels considered to be high will depend on the type of telemetry antenna and lead conductors used and their respective configurations and placement. As such, the MRI detection thresholds will be defined according to the type of IMD system in which MRI detection is being implemented.

At step 215, the IMD telemetry antenna and lead voltage signal sampling rates are set. The sampling rate used for sampling the telemetry antenna voltage may correspond to the signal sampling rate used by the telemetry circuitry for detecting the presence of a telemetry signal received by the telemetry antenna. The circuitry used for sampling the telemetry antenna voltage for MRI detection may correspond to the circuitry used for sampling the telemetry antenna signal for detecting the presence of valid telemetry signals from an external programmer.

The lead voltage signal sampling rate may be set to correspond to the telemetry antenna voltage signal sampling rate. Alternatively, the lead voltage signal sampling rate may correspond to lead voltage sampling performed for other device functions, such as physiological signal sensing, monitoring lead integrity or monitoring for other forms of EMI. As such, the circuitry for monitoring the lead voltage signal for MRI detection may correspond to circuitry included in the IMD for monitoring other signals. Alternatively, dedicated signal sampling circuitry may be provided for sampling the IMD telemetry antenna voltage and/or the lead voltage signals for the purpose of detecting an MRI field.

In some embodiments, the sampling rate of the lead voltage signal may be set to a low rate that is increased in response to detection of an MRI detection threshold crossing by the telemetry antenna voltage, or vice versa. As will be described below, the detection of one or more MRI detection threshold crossings by the antenna voltage signal and/or the lead voltage signal can be used to adjust the sampling rate at step 215.

The telemetry antenna voltage is sampled at step 220 and the voltage signal on one or more lead conductors is sampled at step 223. The sampled IMD telemetry antenna voltages and lead voltages are compared to the corresponding MRI detection thresholds at decision steps 225 and 227, respectively. If a threshold crossing is not detected, the IMD telemetry voltage and the lead voltage signals continue to be sampled at steps 220 and 223, respectively.

If an MRI detection threshold crossing is detected at either step 230 or 233, method 200 proceeds to determine if the MRI detection requirement is satisfied. In one embodiment, if a predetermined number telemetry antenna voltage samples out of a predetermined number of successive samples cross the MRI detection threshold (i.e., M threshold crossings out of N samples), as determined at decision step 230, method 200 proceeds to decision step 235 to determine if the MRI detection requirement has been satisfied. Likewise, if M lead voltage samples cross the lead MRI detection threshold out of N successive samples, as determined at decision step 233, method 200 proceeds to decision step 235 to determine if the MRI detection requirement has been met.

If the antenna voltage samples do not meet the M out of N criteria, method 200 returns to step 220 to continue monitoring the telemetry antenna voltage. If the lead voltage samples do not meet the M out of N criteria, method 200 returns to step 223 to continue monitoring the lead voltage. It is recognized that the predetermined values for M and N may be defined the same or differently for the telemetry antenna voltage and for the lead voltage.

At decision step 235, a determination is made whether the MRI detection requirement has been satisfied. In one embodiment, the MRI detection requirement is defined as both the telemetry antenna voltage and the lead voltage satisfying the M out of N threshold crossings. If only one of decision blocks 230 and 233 is satisfied, the method 200 returns to step 215, where an adjustment to the voltage signal sampling rates may be made. The signal sampling rates may be increased in response to one of the telemetry antenna voltage or the lead voltage meeting the M out of N threshold crossing requirement. Monitoring of the telemetry antenna and lead voltage signals continues at steps 220 and 223.

If both the telemetry antenna voltage and the lead voltage satisfy the M out of N threshold crossing requirements, MRI detection is made at step 240. In other embodiments, additional requirements may be defined in order to detect an MRI field such as correlation between the occurrences of the threshold crossings.

Upon detecting MRI at step 240, an MRI detection signal is generated for use by the IMD microprocessor for controlling an MRI safeguard response. A safeguard response can include any of generating an alarm signal at step 250, implementing an MRI safe mode of operation at step 255, and performing a capture test at decision step 245 to determine if the induced lead voltage is likely to capture the excitable tissue with which an associated electrode is in contact.

An MRI safe mode implemented at step 255 can be a temporary set of operating parameters as will be described in greater detail below. The alarm signal can be an audible sound or perceptible vibration or tissue stimulation generated by the IMD at step 250 that alerts the patient to notify a clinician of the presence of the alarm condition. The clinician will be aware that additional safety measures may be required prior to performing MRI on the patient.

In one embodiment, a capture test performed at decision step 245 includes comparing the sampled lead voltage signal to prior threshold test results stored by the IMD. If the induced energy on the lead associated with the measured voltage exceeds the capture threshold, inadvertent tissue stimulation is likely to occur during the MRI examination.

In an alternative embodiment, a capture test includes delivering a test stimulation pulse at an energy corresponding to the highest lead voltage signal sampled and sensing for a subsequent evoked response. If an evoked response is detected, induced voltage on the lead is likely to cause inadvertent tissue stimulation during the MRI.

A determination that the energy associated with the induced lead voltage is greater than the capture threshold at decision step 245 can cause an alarm signal to be generated at step 250 and/or implementation of an MRI safe operating mode at step 255. If an alarm has already been generated and/or MRI safe operating parameters implemented in direct response to the MRI detection made at step 240, a positive capture test result at step 245 may cause a second alarm to be generated and/or cause supplementary changes to the MRI safe operating parameters. Alternatively, the MRI detection made at step 240 may first cause the capture test to be performed at decision step 245, after which a positive result for the capture test will trigger a patient alarm and/or MRI safe operating mode. A negative capture test result may produce no additional safeguard response.

As such, if the energy associated with the induced lead voltage signals is less than the energy required for capture, method 200 may return to steps 220 and 223 to continue monitoring the telemetry antenna voltage and the lead voltage, respectively. If an induced lead voltage measurement is later found to exceed the capture threshold, an additional safeguard response (patient alarm or implementation of MRI-safe operating parameter settings) can be provided.

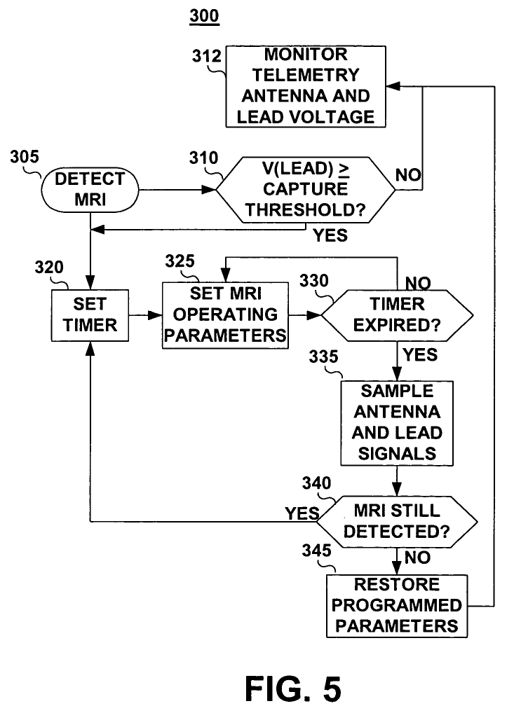

FIG. 5 is a flow chart summarizing steps included in one method for implementing an MRI safe operating mode. An MRI safe operating mode can be implemented after an MRI detection is made at step 305 according to the MRI detection method 200 shown in FIG. 4, or after a positive result of a capture test performed at step 310 in response to the MRI detection at step 305. Implementation of the MRI safe mode begins by setting a timer at step 320 The timer is set at step 320 to a predetermined time interval during which the MRI safe mode will be in effect. The predetermined time interval is generally selected to be at least the length of a typical MRI examination, for example 15 to 20 minutes.

A predetermined set of MRI-safe operating parameters are automatically implemented at step 325. The MRI operating parameter settings may cause IMD therapies to be withheld or modified or may alter physiological sensing performed by the IMD. For example, in the case of a cardiac stimulation device, pacing may be inhibited, cardioversion and/or defibrillation therapies may be disabled or modified or set to a programmed rate, that is inhibiting the sensing operation of the device, and/or arrhythmia detection may be disabled. If the induced lead voltage has been found to be equal to or greater than the capture threshold, the MRI operating mode may include pacing at an overdrive rate to prevent inadvertent capture due to EMI by altering the refractoriness of the heart.

The MRI operating parameter settings remain in effect until the timer expires as determined at decision step 330. Upon expiration of the timer, as determined at decision step 330, the telemetry antenna voltage and lead voltage signals are sampled again. A determination is made at step 340 if an MRI field is still present. Re-detection of the MRI field may be based on the same MRI detection requirement used for the original MRI detection as described above in conjunction with FIG. 4. Alternatively, a less stringent requirement may be defined for re-detecting the MRI field. For example, fewer MRI detection threshold crossings may be required to confirm the continued presence of the MRI field.

If the MRI field is still detected at decision step 340, the timer is reset at step 320 and the MRI operating parameters remain in effect for another time interval. If the MRI field is not detected at decision step 340, the permanently programmed parameter settings are restored at step 345. Telemetry antenna voltage and lead voltage monitoring resumes at step 312.

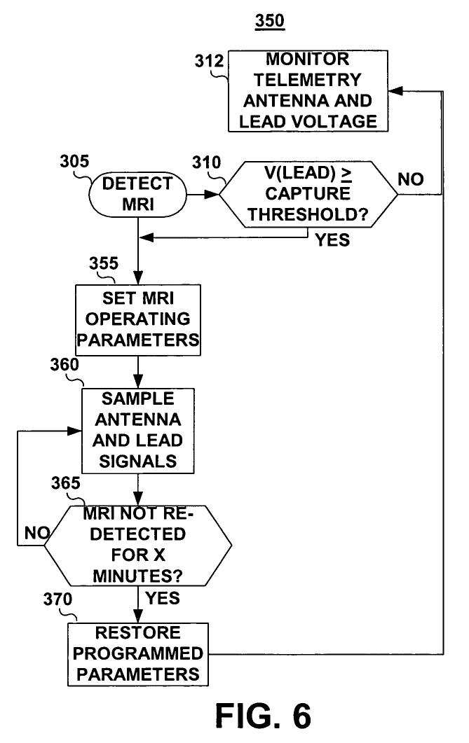

FIG. 6 is a flow chart summarizing steps included in an alternative method for implementing MRI-safe operating parameters in response to an MRI detection. In some embodiments, a timer is not used for timing the duration that the temporary MRI-safe operating mode is in effect. Alternatively, telemetry antenna voltage and lead voltage monitoring continues during the temporary MRI-safe operating mode. Permanently programmed parameters are restored as soon as the MRI field is no longer detected or after a predetermined interval of time during which no MRI re-detection is made.

In method 350, MRI operating parameters are implemented at step 355 in response to an MRI detection made at step 305 or a positive capture test result at step 310. Telemetry antenna and lead voltage signals continue to be sampled at step 360 during the MRI-safe mode. At decision step 365, method 350 determines if the sampled signals meet an MRI re-detection requirement. If MRI is not redetected for a predetermined interval of time, the permanently programmed parameters are restored at step 370. Method 350 returns to step 312 to continue monitoring telemetry antenna and lead voltage signals. If the MRI re-detection requirement is met at decision step 365 within the predetermined interval of time, the MRI-safe operating parameters remain in effect, and antenna and lead voltage signals continue to be sampled at step 360.

Thus, an IMD and associated method for detecting EMI associated with an MRI environment and for providing an automatic safeguard response have been described. The detailed embodiments described herein with the accompanying drawings are intended to illustrate exemplary embodiments of the invention. It is recognized that one having skill in the art and the benefit of the teachings provided herein may conceive of variations to the exemplary embodiments. The described embodiments are therefore not intended to be limiting with regard to the following claims.

1. An implantable medical device (IMD), comprising:
a telemetry circuit antenna;
a lead having an elongated body for carrying a conductor extending from a proximal connector to a distal electrode;
a circuit for measuring a voltage induced on the telemetry circuit antenna and generating an antenna voltage signal corresponding to the measured voltage on the antenna;
a circuit for measuring a voltage induced on the lead conductor and generating a lead voltage signal corresponding to the measured voltage on the lead; and
processing circuitry for receiving the antenna voltage signal and the lead voltage signal and for generating an MRI detection signal if the antenna voltage signal exceeds a first predetermined MRI detection threshold and the lead voltage signal exceeds a second predetermined MRI detection threshold; and
control circuitry for providing a response to the MRI detection signal.

2. The IMD of claim 1, further comprising an alarm actuated by the response from the control circuitry.

3. The IMD of claim 1, further comprising an MRI operation module actuated by the response from the control circuitry.

4. The IMD of claim 3, wherein the MRI operation module inhibits the delivery of electrical stimulation.

5. The IMD of claim 3, wherein the MRI operation module inhibits the IMD from sensing cardiac parameters.

6. The IMD of claim 1, wherein the processing circuitry samples the measured voltage on the antenna at a first rate and the measured voltage on the lead at a second rate and alters the first rate if the second predetermined MRI detection threshold is exceeded.

7. The IMD of claim 1, wherein the processing circuitry samples the measured voltage on the antenna at a first rate and the measured voltage on the lead at a second rate and alters the second rate if the first predetermined MRI detection threshold is exceeded.

8. An implantable medical device (IMD) comprising:
means for providing electrical stimulation;
means for delivering the electrical stimulation to a site;
means for providing telemetric communication including an antenna;
means for measuring a first voltage on the means for delivering and a second voltage on the antenna and proving an output signal; and
means for initiating an MRI response based upon the output signal.

9. The IMD of claim 8, wherein the means for initiating includes means for generating an alarm.

10. The IMD of claim 8, wherein the means for initiating includes means for inhibiting a therapy.

11. The IMD of claim 8, wherein the means for initiating includes means for altering a therapy.

12. The IMD of claim 8, wherein the means for initiating includes means for inhibiting a sensing function of the IMD.

13. The IMD of claim 8, wherein the means for initiating includes means for performing a capture test at the first voltage and determining if a subsequent response is required based upon whether the first voltage causes capture.

14. A method comprising:
sensing a first voltage on a lead coupled with an implantable medical device (IMD);
sensing a second voltage on an antenna coupled with the IMD;
comparing the first voltage to a first threshold;
comparing the second voltage to a second threshold; and
initiating an MRI response if the first voltage exceeds the first threshold or the second voltage exceeds the second threshold.

15. The method of claim 14, wherein the MRI response is only initiated if both first voltage exceeds the first threshold and the second voltage exceeds the second threshold.

16. The method of claim 14, wherein the MRI response includes initiating an alarm.

17. The method of claim 14, wherein the MRI response includes inhibiting a therapy.

18. The method of claim 14, wherein the MRI response includes inhibiting a cardiac sensing function of the IMD.

19. The method of claim 14, wherein the MRI response includes altering a therapy.

20. The method of claim 14, wherein the MRI response includes performing a capture test at the first voltage and taking further action if first voltage captures.
